(19) United States
(12) Patent Application Publication
(10) Pub. No.: US 2018/0108433 A1
(43) Pub. Date: Apr. 19, 2018

(54) SYSTEM AND COMPUTER PROGRAM FOR ANALYZING AND MANAGING HEALTH, FITNESS AND NUTRITIONAL WELLNESS

(71) Applicant: James J. Mahoney, Southlake, TX (US)

(72) Inventor: James J. Mahoney, Southlake, TX (US)

(73) Assignee: CENTER U, L.L.C., Southlake, TX (US)

(21) Appl. No.: 15/844,606

(22) Filed: Dec. 17, 2017

Related U.S. Application Data
(63) Continuation-in-part of application No. 13/933,277, filed on Jul. 2, 2013.

(51) Int. Cl. G16H 10/60; G16H 50/30; G16H 20/60

(52) U.S. Cl. CPC G16H 10/60; G16H 20/60; G16H 50/30

(57) ABSTRACT

A system and computer program product for serving web pages offering fitness and nutritional wellness information. The system includes a plurality of remote computers in communication with a respective plurality of remote users, a central server having a computer program stored in non-transient memory and one or more microprocessors, a network interface in communication with the central server and the plurality of remote computers over a network, and a shared database in communication with the central server. The network interface is configured to receive patient information. The central server is programmed by means of the computer program to receive configuration and setup information, wherein the configuration and setup information includes configuration of user configurable fitness and wellness questions derived from validated questionnaires in a plurality of subspecialty fields, and user configurable correlation rules associated with the fitness and wellness questions, receive and store patient information in the database, wherein the patient information includes responses to the user configurable fitness and wellness questions, correlate the responses based on the user configurable correlation rules to a plurality of functional scores using the one or more microprocessors, provide a health and wellness recommendation based on the functional scores, and transmit one or more web pages, via the network interface, representing the functional scores within the user configurable thresholds, and display the one or more web pages on respective displays of one or more of the plurality of remote computers in communication with respective one or more of the plurality of remote users. The user configurable correlation rules include one or more predefined thresholds. The user configurable threshold includes an upper threshold value and a lower threshold value. Functional scores greater than the upper threshold value are assigned a standard maximum value in the health and wellness recommendation. Functional scores less than the lower thresholds are not included in a final health and wellness recommendation.

### CROSS REFERENCE TO RELATED APPLICATIONS

The present application is a continuation-in-part of and claims priority to U.S. patent application Ser. No. 13/933,277, entitled “System and computer program for analyzing and managing fitness and nutritional wellness,” filed in the U.S. Patent and Trademark Office on Jul. 2, 2013, having at least one common inventor as the present document and hereby incorporated by reference.

### BACKGROUND OF THE INVENTION

### Field of the Invention

The present invention is generally related to systems for health, fitness and nutritional wellness, and more particularly to a system and computer program for interpreting and managing health, fitness and nutritional wellness.

### DISCUSSION OF THE BACKGROUND

Pioneers in functional medicine began research in the early 1950′s into what was called molecular nutrition. The concepts of biochemical individuality and biomolecular psychiatry revealed the integral relationships of nutrition, thought and physiology.

Contemporary health practitioners receive little formal education in functional medicine and molecular or clinical nutrition. Subsequently, few doctors recognize the impact of incomplete nutrition and fitness on the optimal health of their patients. Systematically addressing nutrition and health is a time intensive task that exceeds the abilities of most health professionals.

Inconsistent guidance results from this lack of systematic analysis. Health, nutrition and preventive care require specific diet, nutrition, exercise and remedial lifestyle recommendations.

Age, gender, genetic code, environment and lifestyle factors strongly influence proper selection of nutrition and wellness prescription. These factors must all be included while considering overriding disease states that may be mitigated or eliminated through a health and wellness program.

At least seventy percent of consumers do not discuss nutritional support products with their health professional and miss the benefits of a properly integrated nutritional/medical plan. Potential interactions and side effects of nutritional products with conventional medical care are also eliminated from critical discussion.

The beneficial impact of health, wellness and nutrition has been clearly demonstrated through peer reviewed scientific study. Validated questionnaires have become the “state of the art” in clinical decision making alongside relevant objective clinical data such as blood tests, electrocardiograms and MRI scanning. Current peer-reviewed science validates the following four factors that make a nutritional approach to functional medicine a foundational aspect of healthcare today: (i) nutrition is an environmental factor that influences gene expression and phenotype in each individual, (ii) nutrients act as important biological response modifiers and control or regulate function of tissues, glands, organs and systems throughout the body, (iii) the molecular environment of the body depends on the interaction of an individual's genes with macronutrients, micronutrients, and conditionally essential nutrients, and (iv) diseases such as cardiac disease, adult-onset diabetes, arthritis, digestive disorders, loss of cognitive function and many forms of cancer are often the result of an amalgamation of multiple factors including nutritional under-consumption, poor dietary, lifestyle, fitness and environmental choices.

Use of configurable questions having subspecialty fields and configurable correlation rules is also lacking in the prior art. For example, the present invention is distinguished from U.S. Patent Publication No. 2008-0195594, which, amongst other things, does not teach or suggest configuration and setup information that includes configuration of user configurable fitness and wellness questions derived from validated questionnaires in a plurality of subspecialty fields, and user configurable correlation rules associated with the fitness and wellness questions.

Thus, there currently exist deficiencies in interpreting and managing health, fitness and nutritional wellness in the prior art.

### SUMMARY OF THE INVENTION

Accordingly, one aspect of the present invention is to provide a system for serving web pages offering fitness and nutritional wellness information. The system includes a plurality of remote computers in communication with a respective plurality of remote users, a central server having a computer program stored in non-transient memory and one or more microprocessors, a network interface in communication with the central server and the plurality of remote computers over a network, and a shared database in communication with the central server. The network interface is configured to receive patient information. The central server is programmed by means of the computer program to: (i) receive configuration and setup information, wherein the configuration and setup information includes configuration of user configurable fitness and wellness questions derived from validated questionnaires in a plurality of subspecialty fields, and user configurable correlation rules associated with the fitness and wellness questions, (ii) receive and store patient information in the database, wherein the patient information includes responses to the user configurable fitness and wellness questions, (iii) correlate the responses based on the user configurable correlation rules to a plurality of functional scores using the one or more microprocessors, (iv) provide a health and wellness recommendation based on the functional scores, and (v) transmit one or more web pages, via the network interface, representing the functional scores within the user configurable thresholds, and display the one or more web pages on respective displays of one or more of the plurality of remote computers in communication with respective one or more of the plurality of remote users. The user configurable correlation rules include one or more predefined thresholds. The user configurable threshold includes an upper threshold value and a lower threshold value. Functional scores greater than the upper threshold value are assigned a standard maximum value in the health and wellness recommendation. Functional scores less than the lower thresholds are not included in a final health and wellness recommendation.

Yet another aspect of the present invention is to provide a computer program product embodied on a non-transitory computer readable medium for transmitting web pages offering fitness and nutritional wellness information to remote users. The computer program is implemented by one or more processors executing processor instructions, the computer program product comprising: (i) a first computer code for receiving configuration and setup information, (ii) a second computer code for receiving and storing patient information in the database, (iii) a third computer code for correlating the responses based on the user configurable correlation rules to a plurality of functional scores using the one or more microprocessors, (iv) a fourth computer code for providing a health and wellness recommendation based on the functional scores, (v) a fifth computer code for transmitting one or more web pages, via the network interface, representing the functional scores within the user configurable thresholds, and displaying the one or more web pages on respective displays of one or more of the plurality of remote computers in communication with respective one or more of the plurality of remote users, wherein the user configurable threshold includes an upper threshold value and a lower threshold value. The configuration and setup information includes configuration of user configurable fitness and wellness questions, and user configurable correlation rules associated with the fitness and wellness questions. The user configurable correlation rules include one or more user configurable thresholds. The patient information includes responses to the user configurable fitness and wellness questions. Functional scores greater than the upper threshold value are assigned a standard maximum value in the health and wellness recommendation. Functional scores less than the lower thresholds are not included in a final health and wellness recommendation.

### BRIEF DESCRIPTION OF THE DRAWINGS

A more complete appreciation of the present invention and many of the attendant advantages thereof will be readily obtained as the same becomes better understood by reference to the following detailed description when considered in conjunction with the accompanying drawings, wherein:

FIGS. 1A-1B are block diagrams illustrating a system for analyzing and managing fitness and nutritional wellness in accordance with an embodiment of the present invention;

FIGS. 2A-2F are flow charts illustrating a method for analyzing and managing fitness and nutritional wellness in accordance with an embodiment of the present invention; and

FIGS. 3A-3W illustrate an exemplary user interface and computer program for analyzing and managing fitness and nutritional wellness in accordance with an embodiment of the present invention.

### DETAILED DESCRIPTION THE PREFERRED EMBODIMENTS

Referring now to the drawings, wherein like reference numerals designate identical or corresponding parts throughout the several views, preferred embodiments of the present invention are described.

According to one embodiment, predefined thresholds and an easy-to-use professional user interface is utilized to reduce prejudice or inaccuracy. The intuitive interface is configurable to conform to a professional user's practice style or modularity. The system allows substantially all individual practice preferences to be configured, including without limitation how the questionnaire is presented.

The system interview is derived from many questionnaires employed in subspecialty areas of wellness, nutrition, and medicine. This list includes without limitation psychiatry, endocrinology, gynecology, urology, neurology, gastroenterology, acupuncture, metabolic medicine, rheumatology, oncology, cardiology, osteopathy, orthopedics and psychology.

The questions though asked only once apply to many categories and are overlaid to display impact on global health categories rather than disease conditions (diagnoses). The questionnaires include a minimized number of questions as each one applies to multiple health specialties. Reduced redundancy improves analysis and minimizes errors. Each of the responses to the questionnaires have a configurable numerical functional score for each specialty that is impacted. For example, a “yes” response to the question “Do you have a headache?” supports the possibility of depression, thyroid or orthopedic problems. Each additional point(s) in favor of that specific concern increases the probability score that the affected system is indeed the causative factor. The questions presented and the numerical functional scores associated with the responses are infinitely configurable. A rules-based engine analyzes the questionnaire results and based on the configurable rules, arrives at an overall numerical functional score for each category. Reports, graphical representations and recommendations are then provided to the patient or user. After the preliminary report is reviewed the professional updates and signs the interpretive medical record adding physical examination data and further in-depth interview data as elicited from the patient or user. Historical information from questionnaires and laboratory data is maintained so that trend analysis can be performed. A sequential graphic improvement score and/or trend chart may be presented showing the user's improvement over time. The system identifies risk factors and may be configured to send an automated warning communication to the user via email, text and other means if a risk threshold is exceeded.

The system may be configured to assist the doctor in screening and/or prescreening patients. The professional user can upload and/or add comprehensive notes for a medical treatment plan.

The system includes a nutritional supplement store to address needs linked to outputs from individual questions as well as rules-based algorithm-derived multisystem applications.

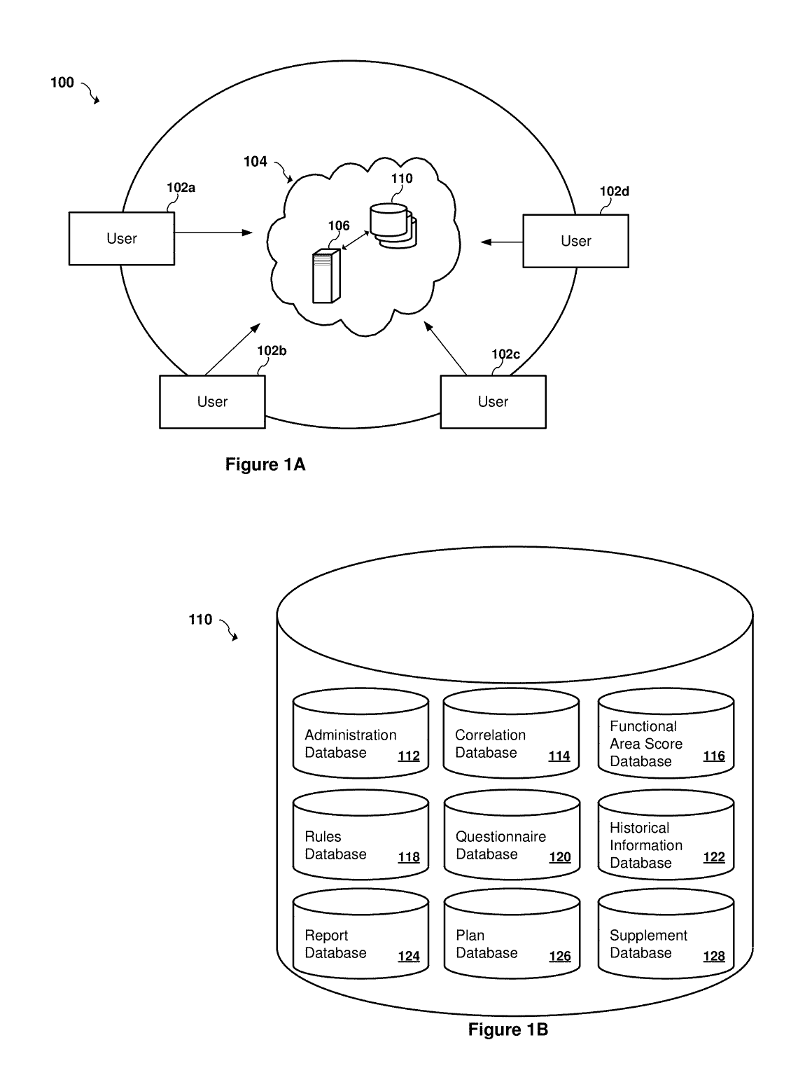

Referring to FIGS. 1A-1B, block diagrams illustrating a system 100 for analyzing and managing fitness and nutritional wellness in accordance with an embodiment of the present invention is shown. The system 100 includes one or more computers and/or servers 106 in communication with one or more databases 110. According to one embodiment, the one or more databases 110 include without limitation an administration database 112, a correlation database 114, a functional score database 116, a rules database 118, a questionnaire database 120, a historical information database 122, a report database 124, a plan database 126, and a supplement database 128. It is understood, however, that other databases and/or combinations thereof are possible within the scope of the present invention. According to one embodiment, one or more patients or users (102a-102d) using an interface device may access the system 100 via a network 104. The interface device may include without limitation a computer, laptop, PDA, smart phone, server, and the like. The network may include without limitation the Internet, Intranet, LAN or WAN, and the like. The one or more users (102a-102d) may utilize a web browser or other software to communicate with the system 100. According to an alternate embodiment, the system 100 is a standalone version loaded on a computer of the one or more users (102a-102d), which may or may not include a network.

The system 100 receives input data from various sources, including without limitation input directly from an interface device (not shown) by a patient or user, a professional user/clinician, or a laboratory or diagnostic provider. A system 100 may be accessed via a secure website by the interface device through a socket of a secure encrypted server. Once the website is accessed, the patient or user is presented with a set of dynamically changing questions in response to answer selection and trends toward increased probability.

The types of input data that may be received into the system 100 include but are not limited to the types of described in Table 1 below.

TABLE 1
EXEMPLARY INPUT DATA

| TYPE | DESCRIPTION |
|---|---|
| Demographics | Information that distinguishes a user |
| | including data points like age, |
| | weight, height, gender etc. |
| Family History | Information pertaining to historical |
| | family environmental, medical, |
| | physiological, and/or psychological conditions. |
| Personal History | Information pertaining to historical, |
| | personal, environmental, medical, |
| | physiological, and/or psychological conditions. |
| Social History | Information regarding specific |
| | socio-environmental, sociological, and |
| | various social conditions that a person |
| | currently is, or has been exposed |
| | to in the past. |
| Current | Information that pertains to the |
| Supplements | supplements that are currently being |
| Utilized | utilized by the user. |
| Lifestyle | Information that pertains to a person's |
| | habitual lifestyle patterns |
| | including, but not limited to habitual |
| | thoughts and mental outlook, |
| | exercise, fitness, eating habits, smoking habits, |
| | and alcohol consumption etc. |
| Body's Red Flags | Information regarding a person's perceived |
| | and objective measures of |
| | well-being including emotional, global |
| | and specific physical, and |
| | global and specific mental measures. |
| Oriental | Physical characteristics and behavioral |
| Medicine | patterns suggest specific organ |
| Biopsychotype | system dysfunction and failure along |
| | predictable lines according to |
| | well-documented principles of Eastern |
| | medicine. The system identifies |
| | these patterns and directs users and |
| | physicians to scrutinize these |
| | specific organ systems for their possible |
| | contribution to health diminishment. |

In addition to patient or user generated data, the system 100 is integrates data from clinical examination procedures and laboratory studies. Clinical and laboratory data may also be input through a secure socket connection of the secure server or through an electronic data interface with a laboratory or clinic. Clinical data is defined as any information gathered by all means through a direct encounter with the patient or user. Laboratory data includes all information generated by any process of collecting and assessing biochemical metabolites, markers, laboratory values, and analytical data including, without limitation, salivary or pH tests; blood, blood plasma, or blood spot tests; urinary tests; stool samples; and caliper readings.

Additionally, the system 100 is capable of utilizing data from biometric measurement devices. Biometric data may include, but is not limited to: EKG data; EEG data; biometric scan data, digitized image data, electromagnetic feedback reader data, and electronic energy flow detection device data. At least a portion of the input data is entered into a correlation database 114 of an expert system engine.

System reporting may be utilized to improve quality of life and genetic expression as well as identification and action to mitigate the impact of disease. A goal of the system is to modify the functional genetic expression evidenced through improved laboratory parameters, sense of well-being, and objective physical and behavioral parameters. Output data may include, but is not limited to the output data shown in Table 2 below.

TABLE 2
EXEMPLARY OUTPUT DATA

| TYPE | DESCRIPTION |
|---|---|
| Lifestyle Plan | Customized lifestyle tips shown to address |
| | pertinent issues the user is facing. |
| Precision | Stretching, cardiovascular, and |
| Fitness Plan | resistance training plans specifically |
| | designed according to the users exercise |
| | history, ability, and goals. |
| | Maximal and training heart rate, |
| | duration, method, type of exercise, and |
| | precise rest periods are among the |
| | essential components that produce |
| | benefit in this plan. |
| Precision | An eating plan that determines the |
| Eating Plan | particular carbohydrate to protein |
| | ratio for the user determined by |
| | calculation of specific inputs the user |
| | provided the system. The eating plan |
| | uses different foods and feeding |
| | schedules to maximize endogenous |
| | homeostatic control on multiple levels. |
| Nutrition Plan | An individualized compilation of |
| | foods to eat that will benefit the |
| | individual as well as a compilation |
| | of foods to avoid due to their |
| | negative impact on digestion, |
| | physiology, symptoms or |
| | digestive/allergic properties is a |
| | significant contributor to the effect of |
| | the plan. Blood type, allergy, |
| | underlying functional challenges and |
| | food preferences are integrated into the system. |
| Nutritional | Function-enhancing and supporting |
| Supplements Plan | nutrients and agents targeted to a |
| | unique profile (individually and |
| | combined in defined proportions). |
| Nutritional | The ingredients in each supplement that |
| Supplement | the user is currently consuming |
| Inventory | is provided. These ingredients are |
| Data | compared with independently lab |
| | certified or otherwise third-party verified |
| | supplement data. The data |
| | provides specific information allowing |
| | the user to make an informed |
| | decision regarding the continuation |
| | of specific supplements the user is |
| | currently taking, or make an |
| | informed decision regarding the |
| | discontinuation of specific supplements |
| | that are no longer necessary, as |
| | well as verify the importance of |
| | additional supplements that may be |
| | added to establish or more thoroughly |
| | complete the clinical and peer- |
| | reviewed observations of the system. |
| Precision | Using well-established methods of |
| Emotional | distraction (stretching and other |
| Repair Kit Plan | physical activity) from a troubling |
| | emotion-evoking stimulus (i.e., the |
| | thought of spiders), the user participates |
| | in precision emotional repair |
| | by speaking affirmations that are targeted |
| | to the specific fear as well as |
| | other emotions that are likely to be |
| | present based on input data derived |
| | from the questionnaire. These areas |
| | of emotional need are largely |
| | derived from a bio-psychotype model |
| | of oriental medicine in addition |
| | to self-reported areas of need by the user. |
| Doctor's | Information includes additional |
| Suggestions | supplements, lab tests, and therapies |
| | designed to give the user's physician |
| | a variety of doctor assisted |
| | remedies or methodologies to more |
| | accurately address the user's needs. |
| Drugs and | Drugs and other pharmaceutical preparations. |
| Pharmaceutical | |
| Preparations | |
| Biologicals | Vaccines and sera. |
| Gene modifying | Treatments which modify genes |
| treatments | and/or genetic expression. |
| Therapies | Various physical, emotional, |
| | energetic, and mental therapies |

A trend represents measurable or perceived changes in the user after implementing one or more plans or utilizing the information produced in output data to initiate healthful changes in indicated areas. A trend may identify behavior patterns that improve disease avoidance, changes in symptoms suggestive of disease, and individually-reported subjective health status. Trends are not reported until the patient or user enters input data into the process flow system 100 a second or more times.

New input data corresponding to the trend is stored in a historical information database 122. Output data generated by the expert system engine is also stored in the historical information database 122. The historical information database 122 allows the trends and output data to be stored and analyzed.

System 100 may be used and accessed by the patient or user an infinite number of times reentering input data and generating a new set of output data containing information specific to their current health situation. The data may be stored and trended allowing the process to be repeated with an evolving focus to address current input data.

System 100 includes databases, tools, rules engines, and content generators. Correlation database 114 stores all input data. As previously described, the input data can be generated by the user, by a clinical examination, a laboratory study, or directly from biometric measurement devices.

System 100 includes a rule creation tool, which may be a simple graphic user interface to allow an expert system programmer or administrator to create a correlation rule. Each correlation rule is capable of giving a range of positive, negative, or neutral values to identify the impact of the the input data for each particular functional area. A functional area is directly associated with the generation of health and disease resistance. This might include digestion, elimination, immune activity and brain function as an example. More specifically, a functional area may be related to the user's thyroid performance, anxiety level, and allergies. For instance, if the user identifies a family history of cardiac disease as well as a lifestyle comprised of little exercise and high fat foods, the correlation rule will weigh these combined factors as highly negative, and factor in the remainder of the input data to generate a functional score for cardiac health. The functional score is simply a numerical value given to the health factor after the correlation rule is applied to the input data. A highly negative functional score for cardiac health may also impact the functional score of another health factor and vice versa. Laboratory data included in the final assessment may trump the symptom score by identifying a blockage in a coronary artery that needs urgent surgery. The sensitivity of symptom assessment is far higher than standard medical testing and makes this a very low likelihood phenomenon.

A rules database 118 stores the correlation rules that relate to functional areas. The correlation rules give negative and positive weights to input data that is relevant to a defined functional area. For example, if the user identifies a family history of cardiac disease (a negative indicator for cardiac health), but has a lifestyle comprised of frequent exercise and healthy diet (a positive indicator for cardiac health), the correlation rule will weigh these indicators against the rest of the input data to generate a functional score for cardiac health.

The system 100 applies a correlation rule to the input data stored in the correlation database 114 and calculates the functional score. Additionally, system 100 calculates a baseline value for each functional area. This baseline is the calculation of the highest possible positive value and the lowest possible negative value for each functional area. By comparing this baseline value to the calculated functional score, the system determines whether the positive or negative value is a priority set for the patient or user. The functional score database 116 stores each calculated functional score as well as the relevant baseline value for each functional area. This data is stored every time input data is entered. The functional scores also may be accessed during the trend analysis described above.

The report database 124 may include without limitation text and graphic content data that is entered by the administrator. The report database 124 is a repository of substantive content that is typically utilized in combination with intelligent rules to display health and wellness information to a user. The content data may be developed in a logical fashion without the administrator having to understand the inner workings of the system 100.

The content data may be utilized in the dynamic creation of output data. Content is created by the administrator to address specific patterns or scenarios identified by the system 100. The system 100 retrieves content data (as text or graphical data) to create a customized report for the patient or user based upon the input data, functional scores, and historical trends analysis.

The system 100 may include a rules engine executing rules against input data, functional scores, and trend data to determine the content data for the user. The rules engine is capable of ensuring that specific nutrient information, such as dosages, are listed at the level scientifically shown to address the most complex functional area. For example, if two functional areas are indicated as area of concern, both of which science indicates may be ameliorated with Vitamin C but at different dosages, the rules engine will determine and present the optimal dosage to address both functional areas appropriately.

The system 100 may utilize a rules engine to analyze the functional scores. When a more focused output data is required, multiple intelligent rules engines may be utilized to analyze input data and functional scores. The result being thorough yet narrowed output data directed to the user.

A supplement database 128 stores data on supplements the patient or user is currently taking and their nutritional content. If the user is taking a supplement that is not found in database 128, the user may enter the label information of the supplement.

The patient or user entered label information may be validated and approved by the administrator for accuracy before approval for general use in the database. The supplement database 128 also may be updated by the administrator. Additionally, direct manufacturer data feeds as well as clinic data feeds may be used to populate the supplement database 128, but as with the supplement label data entry tool 218, the supplement data may be validated by an administrator prior to becoming generally available.

The system 100 queries the supplement database 128 for all supplements the user indicated as currently being taken. The system 100 analyzes all the nutrients identified by the user as being consumed, matching them to the nutrients in the supplements currently being taken. A report is created for the user providing information on what supplements the user may consider continuing, what supplements the user may choose to add, and what supplements may not be a priority or may be discontinued based on the functional scores, input data, and physician recommendations.

The system 100 utilizes a plurality of dynamic report generators that assemble the content data to be utilized in the dynamic creation of output data. The dynamic report generators build a graphically pleasing intelligible report for the patient or user that details their individualized plans. The report is dynamically produced at the patient's or user's request and is presented in a report viewer in a standard internet browser. The dynamic nature of report generation allows the patient or user to receive updated output data with each request to view their report. This is accomplished by using the stored input data as well as the stored functional scores to compile a report using the latest updated content data from the report database 124.

A patent or user 102 may access to the system via a secure socket connection to a web server running on the system 100. A web-based graphic user interface allows the user to answer a dynamically changing questionnaire by entering input data. Input data is converted by the system 100 to create output data. The output data is presented to the user in the form of an online report (medical record quality) displayed by the web server over the secure socket connection.

A patient or user 102 may access the system 100 from a doctor's clinic or office as well as any other location with network connectivity. The patent or user 102 may connect via a secure socket connection to a web server executing on the system 100. In this embodiment, a professional/user may also connect via a secure socket connection to add additional input data and/or access the user's output data to approve and/or modify the output data prior to compiling a report for the patient or user. The report is presented to the patient or user 102 in the form of an online report displayed by the web server over the secure socket connection.

A professional/user input tool allows additional information to be added to the system 100 manually. The information may include data such as laboratory data, diagnosis data, and physical evaluation data. The additional information provided by the professional/user is stored in the correlation database 114 and is utilized as input data.

An output manipulator may be utilized by the patient's or user's professional/user to further customize the output data for the user. The output manipulator is accessed by the doctor using a secure socket connection through a web server executing on the system 100. Using the output manipulator, the professional/user can select what data is be presented to the patient or user. For example, the doctor may decide not to present the user with certain diet or exercise guidance because of physical constraints that were observed while evaluating the patient. The doctor is also able to add additional content to the users report via the output manipulator. For example, a prescription or other information may be added by the doctor. The information can then be added and stored in the user's report.

The report database 124 stores the modified output data in viewable and printable forms for future reference by the user or the doctor. The output data is also stored in raw data form to be used for trend analyzes and dynamic adjustments to the expert system engine and to create a customized user experience for future user interactions. Patient's or user's reports are stored in viewable and printable form for secure access.

### Functional Score Calculation

According to one embodiment, a patient's functional score for a particular rule is calculated by dividing the points received for a patient during testing for a rule (x) by the maximum points available for a given rule (y) and multiplying that value by 100 percent (z). If the calculated functional score is above a configured rule threshold (t), then it appears as a hotspot score which is the calculated system dysfunction scores. For example, assuming x=25 and y=32, then the functional score (s) is calculated as follows:

functional score=(x/y)*z=(25/32)×100%=78.125%

According to one embodiment, this value may be rounded to a whole number or in this case 78%. Assuming the threshold (t) for that particular rule is 10%, then the functional score (s) is significantly greater than the threshold (t) and there may be major issues that need to be addressed by the patient or user.

Each patient is presented with a set of multiple questions (100 or more). The answer to each question has an associated numerical value that add to or subtract from the total points for any problem area a person may have. For example, assuming there may be nine questions for blood sugar and the maximum points available for blood sugar rule (y) is sixteen. This means that if the points received for a patient during testing for the blood sugar rule (x) is twelve points, then the functional score (s) would be (x/y)*z=(12/16)×100%=75%. This may be 75% on a scale of 0 to 100 with 100 being the worst possible case, and this functional score may indicate that the patient has medical issues associated with the blood sugar rule.

The serum blood glucose becomes significant when scoring is out of the range of the normal population. If serum blood glucose is high then it adds into the possible total points, thus increasing the ending percentage and if alarmingly (critically) high or out of range it would override all of the patient-answered questions resulting in “Critical Alert” response by the system 100. This would result in system-generated warnings to both professional and end/user.

In a non-limiting example, let's assume that a patient lab reveals a random (non-fasting) blood glucose of 109 mg/dl and is assigned value of negative one (−1) to their blood glucose rule indicating a slightly lower probability of blood sugar issues relating to their health and wellness. Lets also assume that the blood glucose rule is configured to activate an override at levels low and high both influencing “blood sugar” rule. Exemplary blood glucose override values may be defined as: (i) a blood glucose of less then 60 mg/dl is hypoglycemia (low blood sugar) results in a “consult with health professional” override for the medical condition; (ii) a blood glucose of between 60 and 100 mg/dl is within the normal range and is assigned a negative one (−1) point value for the blood sugar rule as it decreases likelihood of blood sugar regulation problems though does not eliminate it; (iii) a blood glucose of between 141 and 200 mg/dl is assigned a positive two (2) point value to the rule as high blood sugar problems are more likely; (iv) values between 201 and 250 is assigned a positive five (5) point value to the rule. A blood glucose above 250 is highly predictive of diabetic risk/disease resulting in a “consult with health professional” override. An override response eliminates the patient's response score resulting in a “Critical Alert” recommending immediate intervention to the exclusion of any action based on symptom questions. For instance, a value of 100% might be automatically assigned to the rule regardless of the patient's response resulting in immediate “Alert” reporting.

The reference values for a normal random glucose test in an average adult in the range of 60-140 mg/dl to 140-200 mg/dl is considered pre-diabetes, and greater than 200 mg/dl is considered diabetes. According to ADA guidelines, the patient should visit their doctor or go to a clinic for additional tests. However, a glucose greater than 200 mg/dl does not necessarily mean you are diabetic.

### Processing Flows

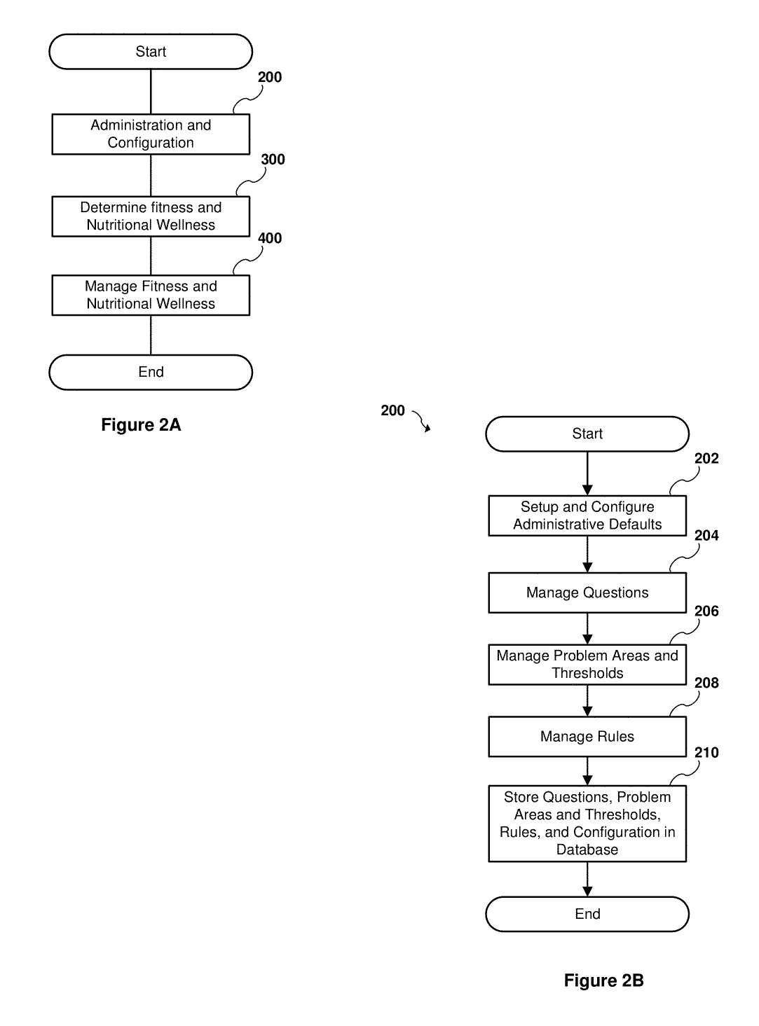

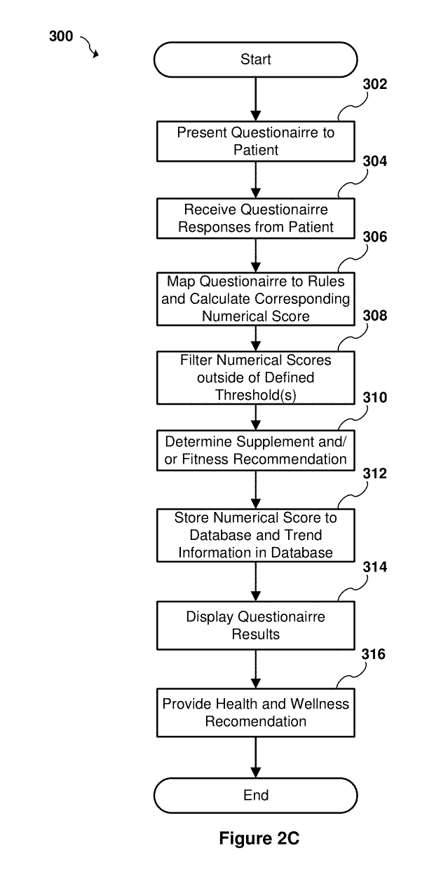

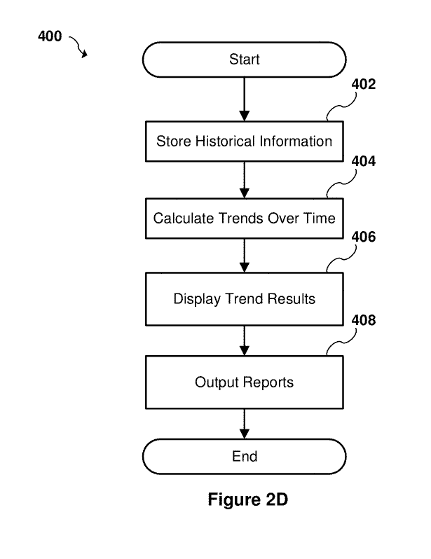

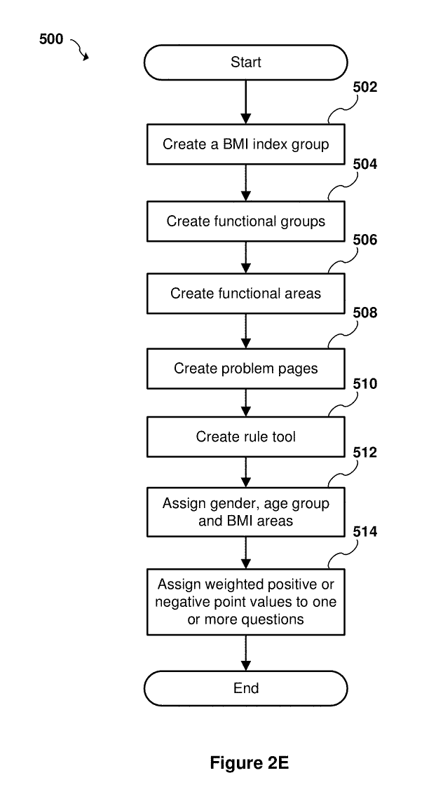

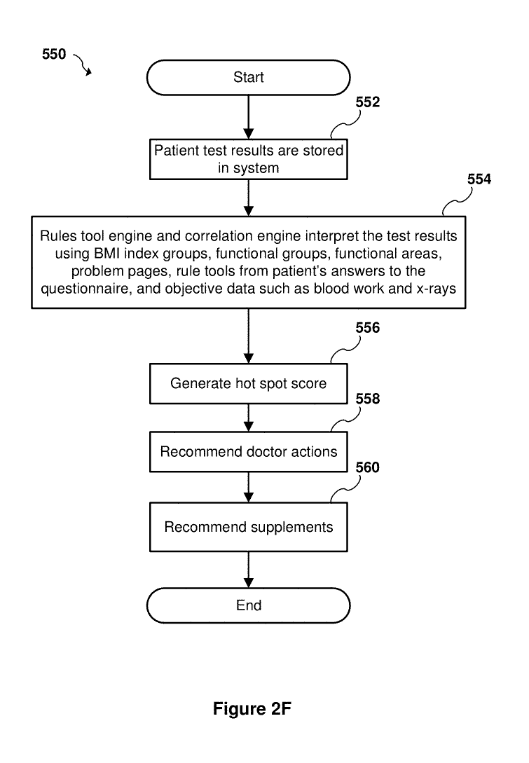

Referring to FIGS. 2A-2F, flow charts illustrating a method for analyzing and managing fitness and nutritional wellness in accordance with an embodiment of the present invention, are shown. The system 100 includes modules without limitation for administration and configuration 200, determining fitness and nutritional wellness 300, and for managing fitness and nutritional wellness 400. These modules may be accessed in any order.

The administration and configuration 200 module is used to setup and configure the system 100. At block 202, the system 100 receives setup and configuration information. Such setup and configuration information may include without limitation branding information, store options, managing sites, loading zip files, autoresponders, and changing passwords. The setup and configuration information also include without limitation managing clients, signup forms, supplements, report options, fitness plans, and eating plans.

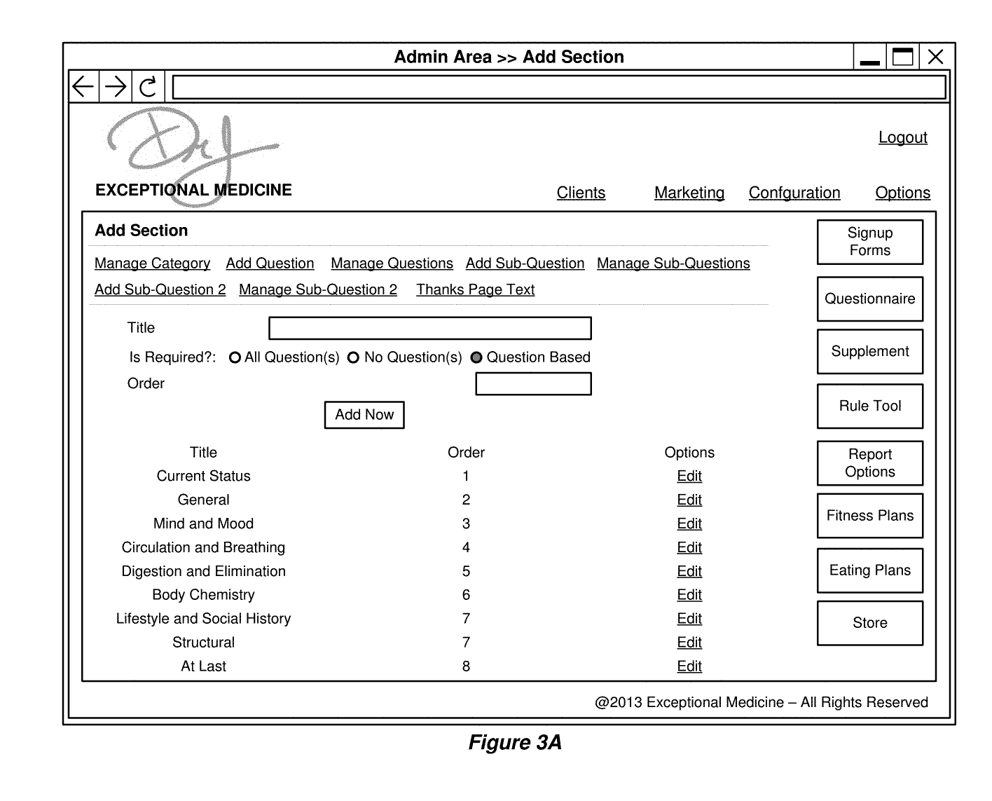

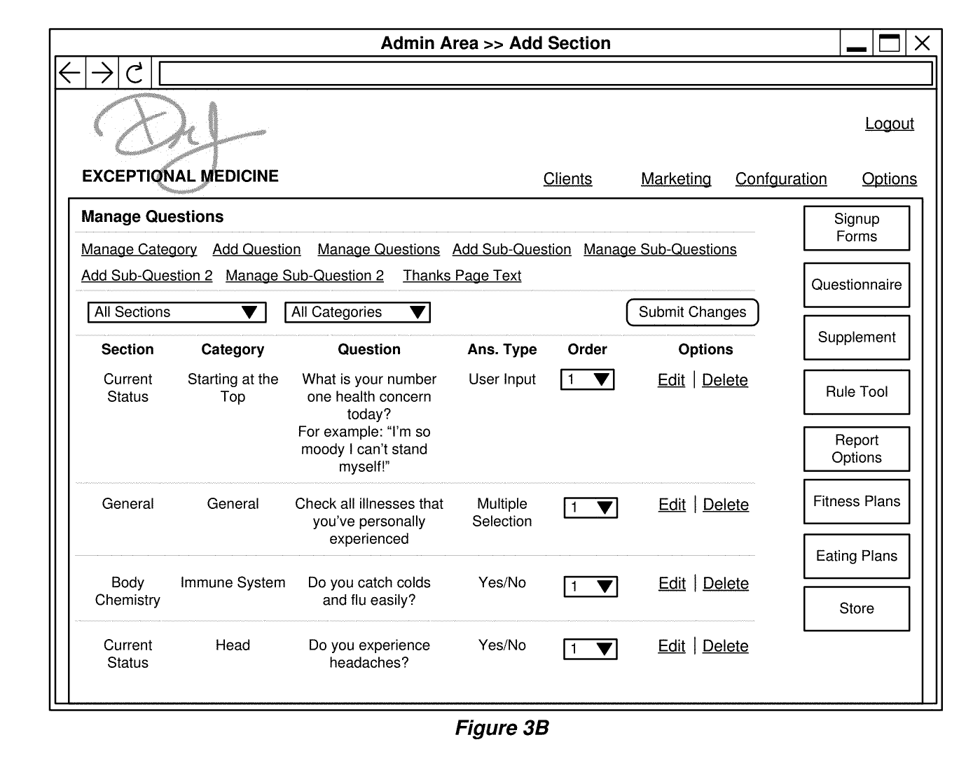

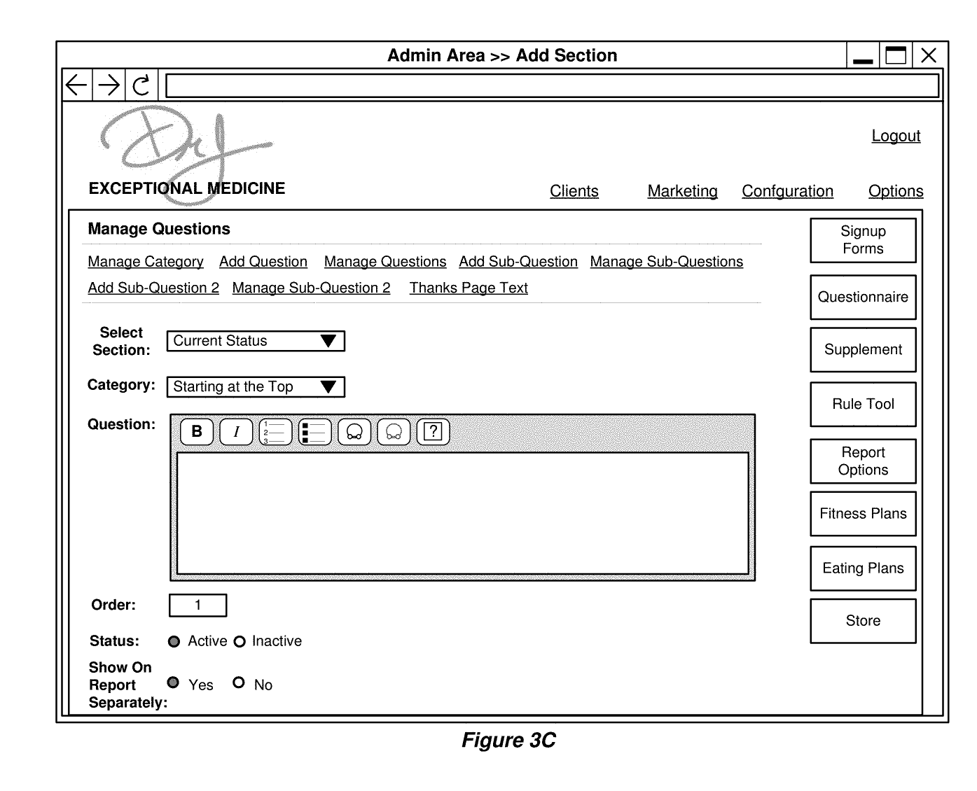

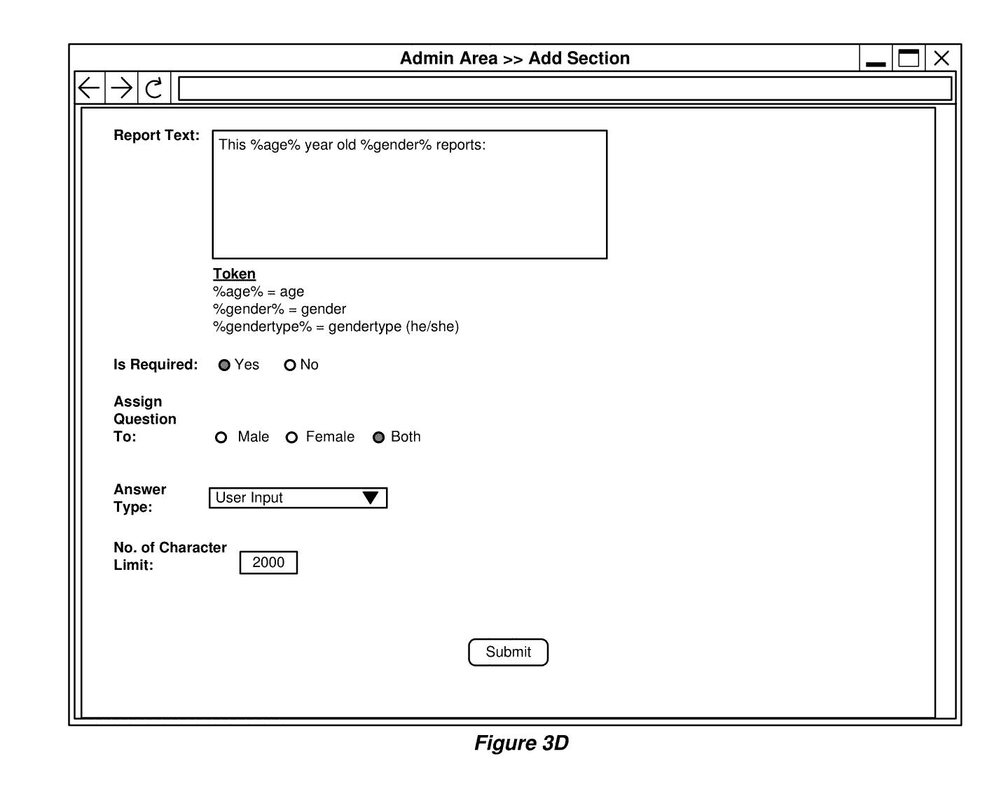

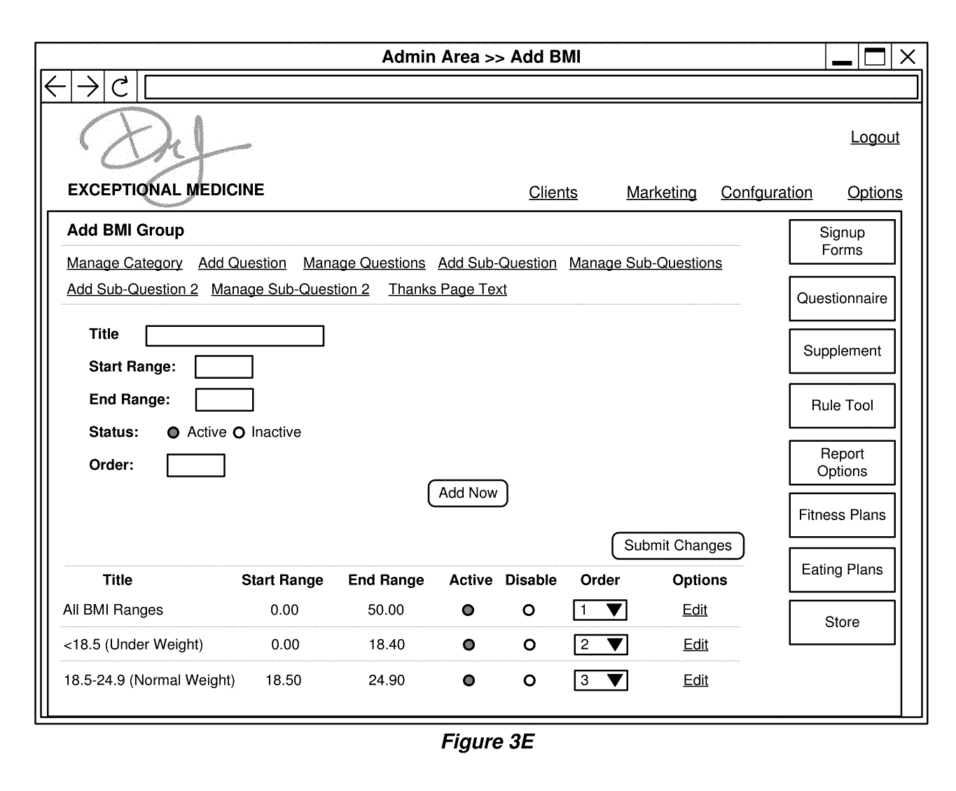

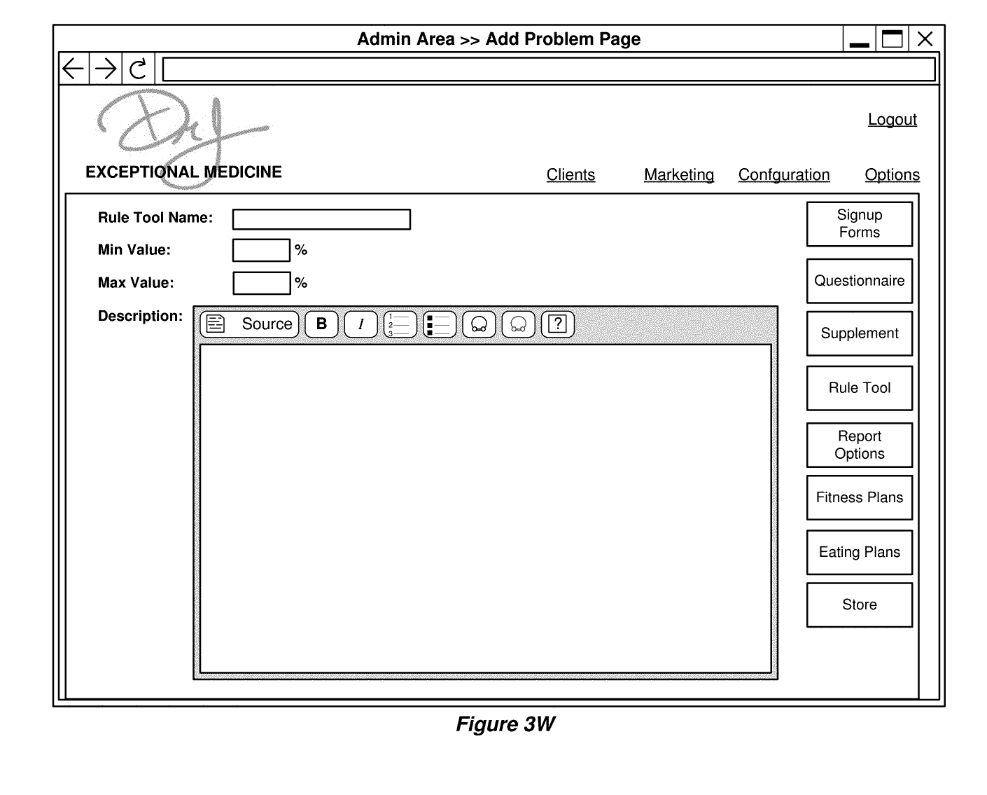

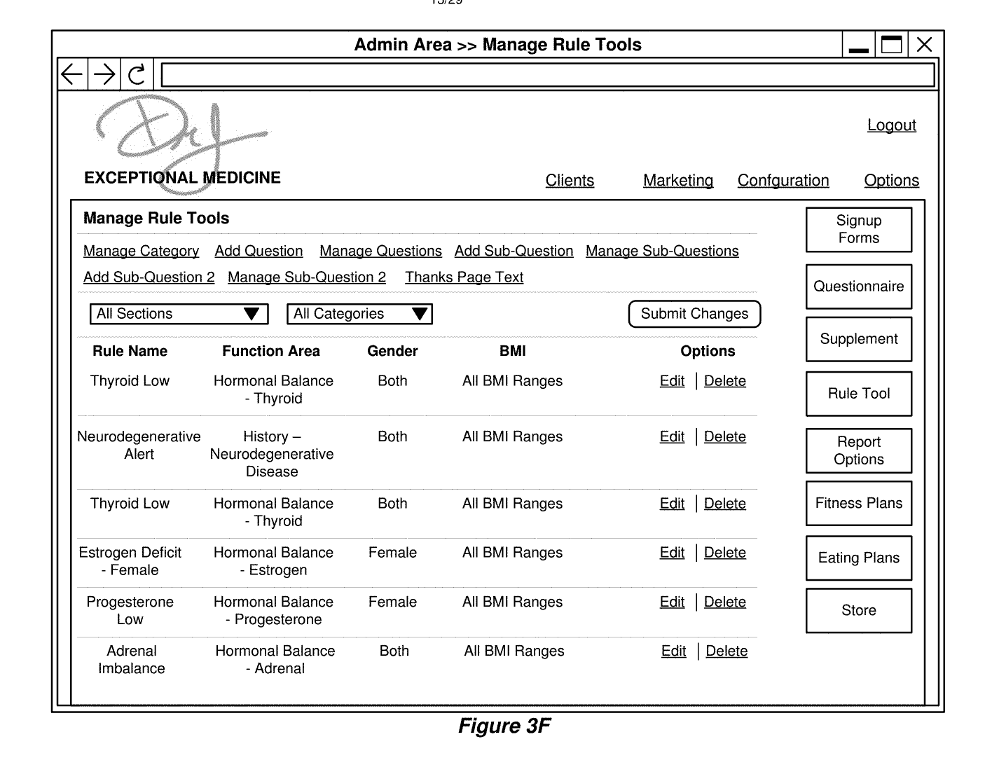

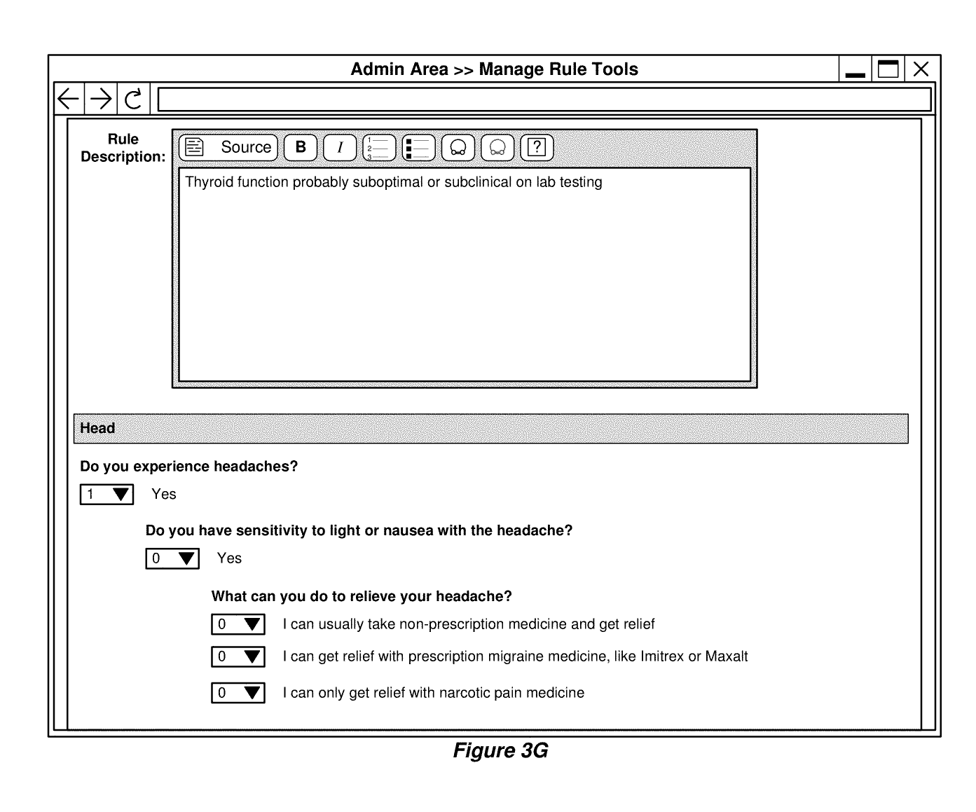

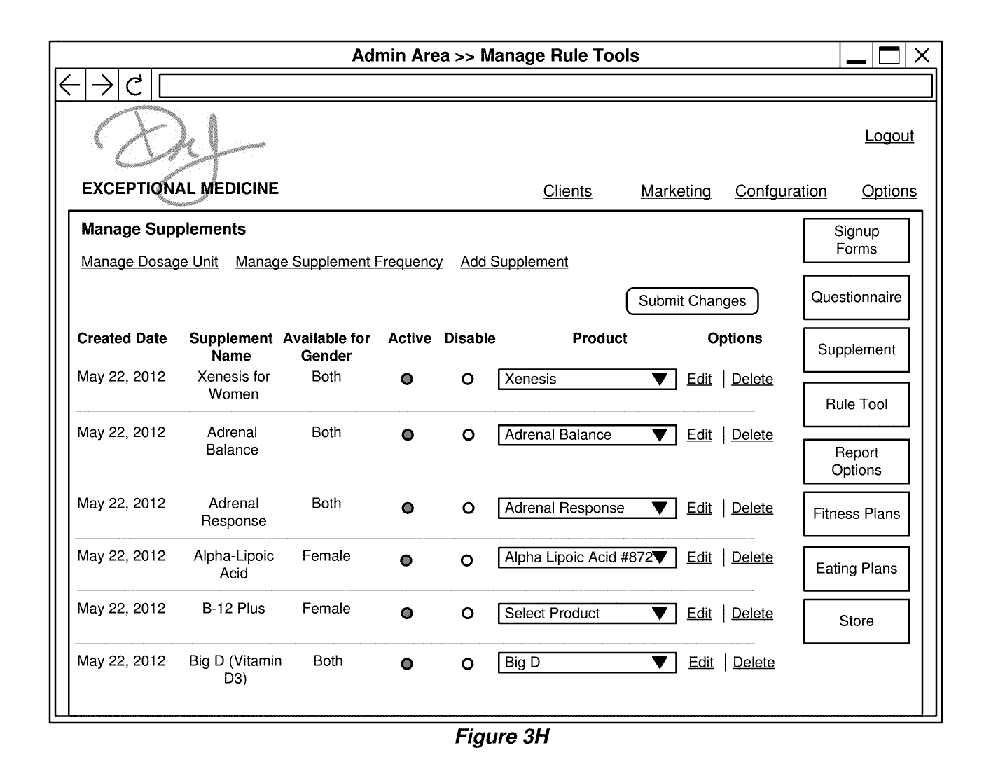

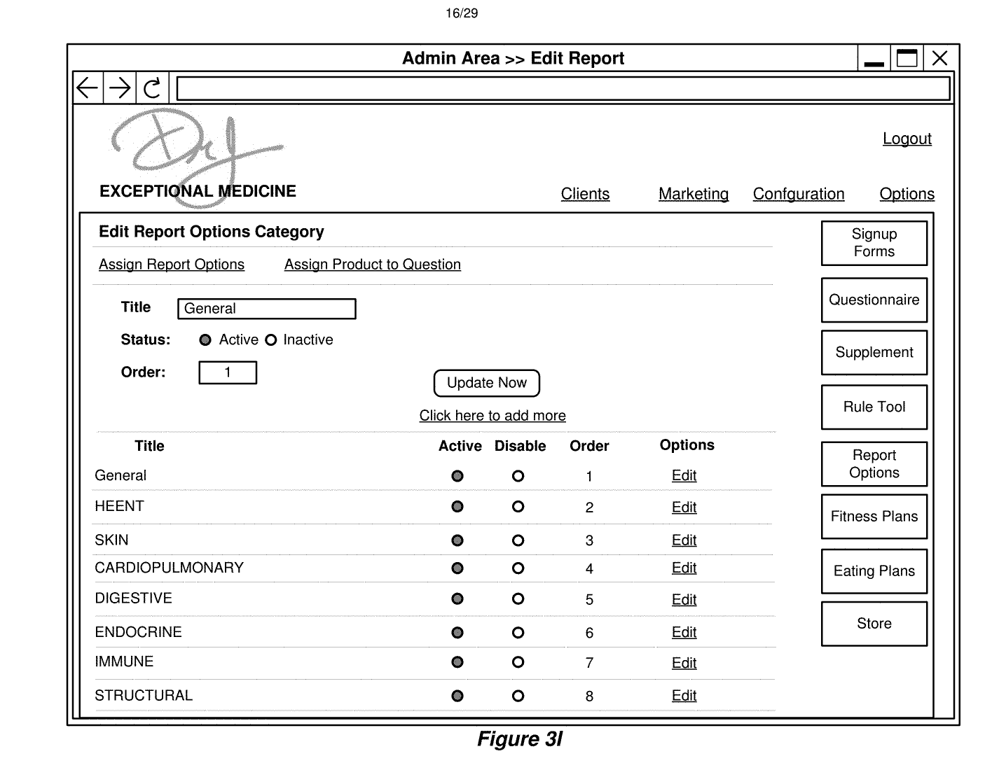

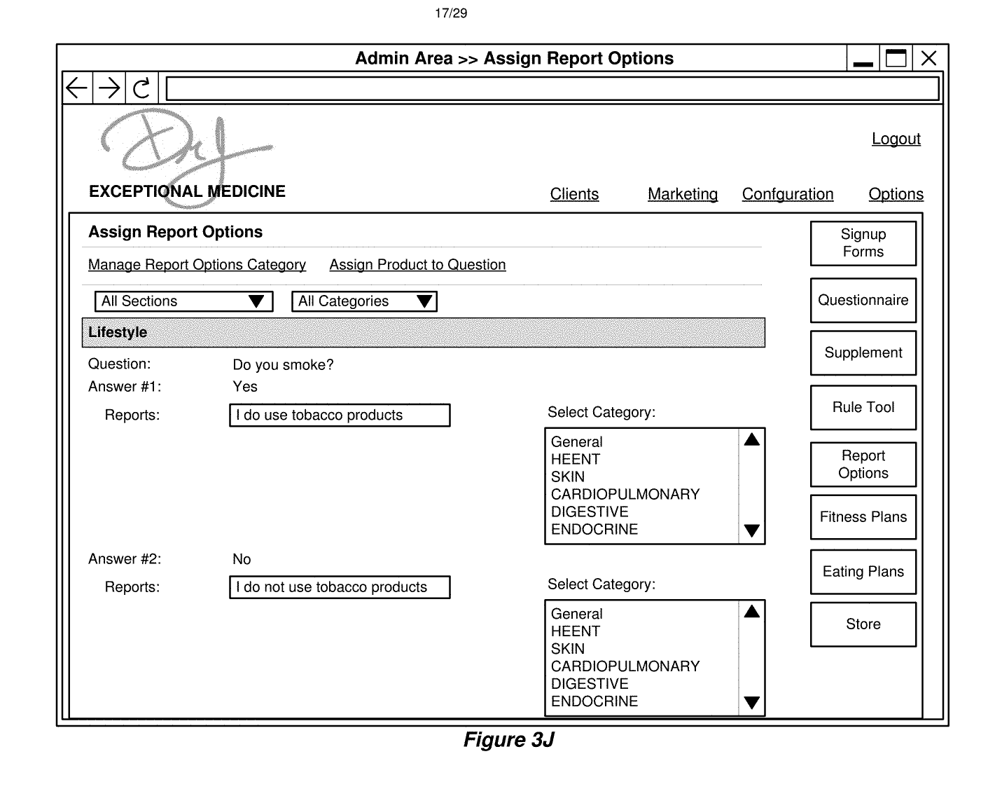

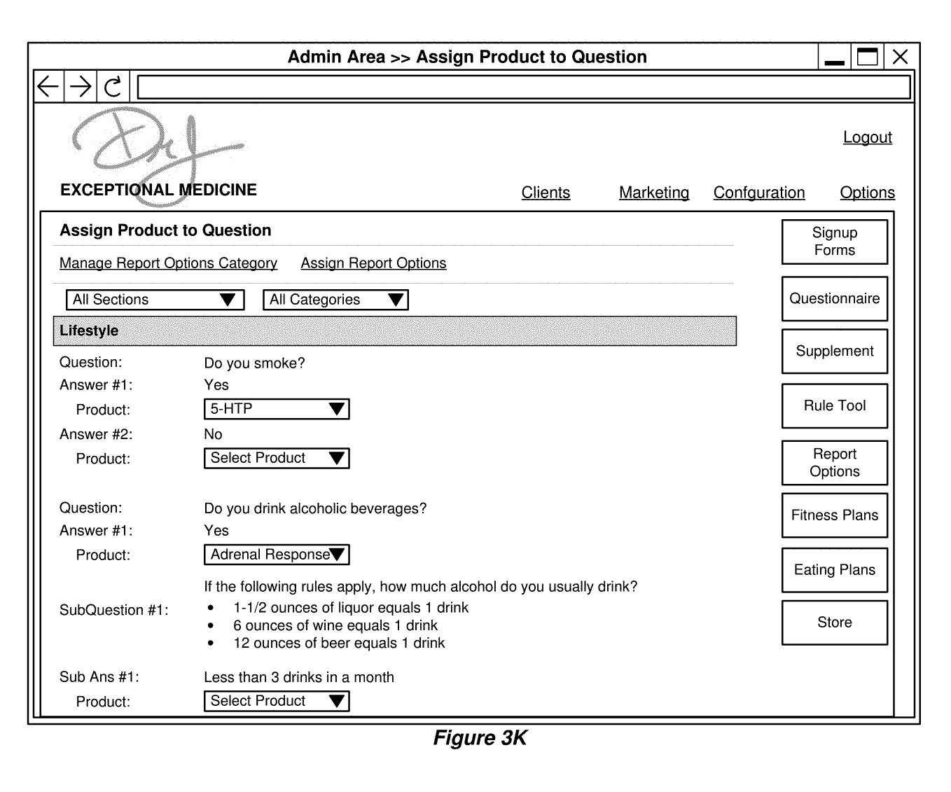

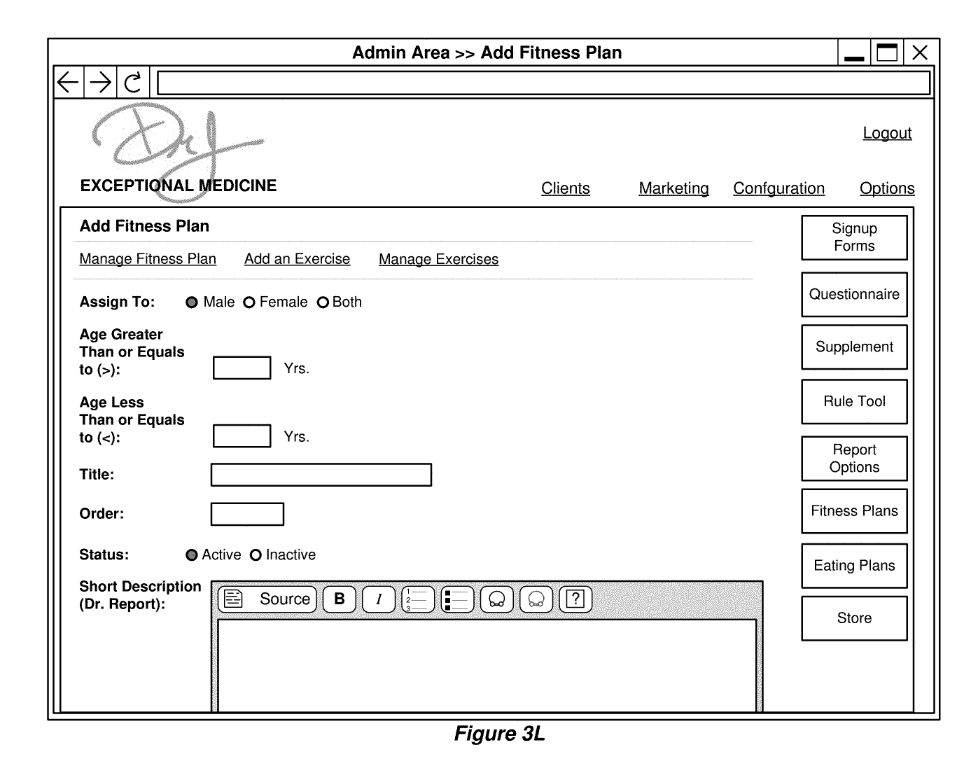

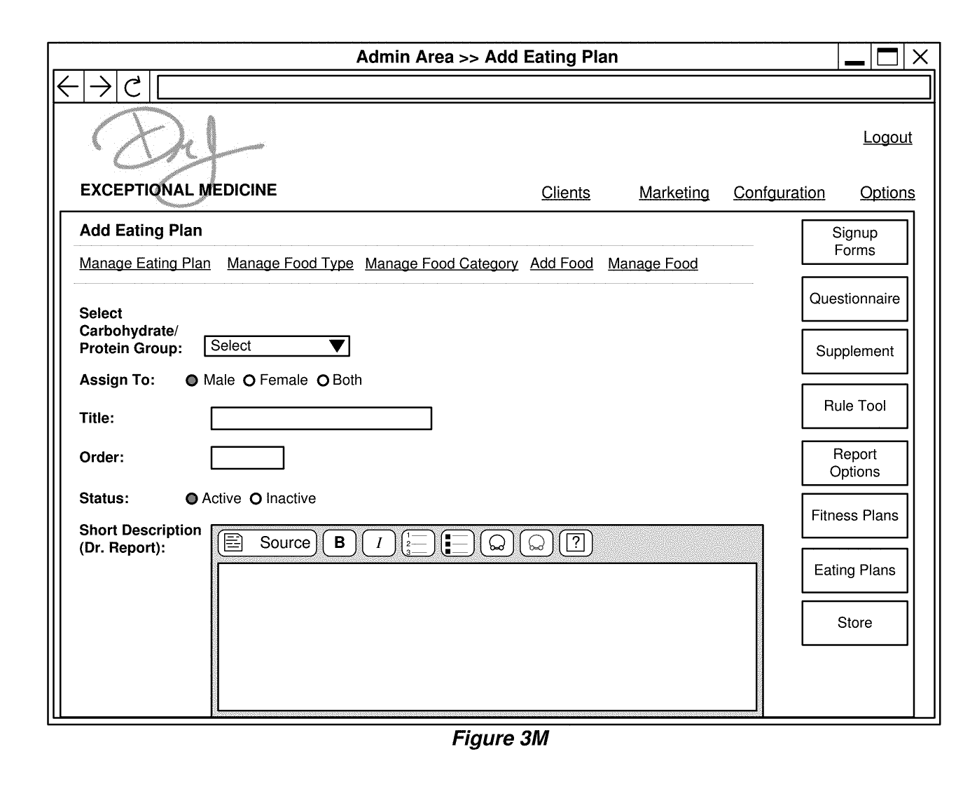

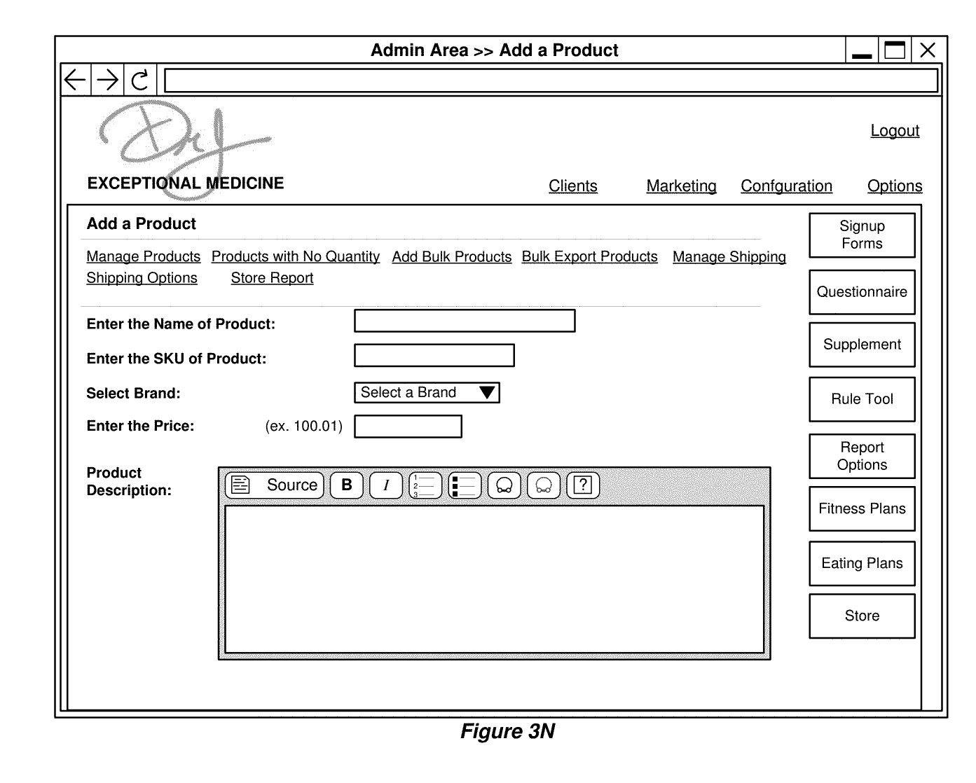

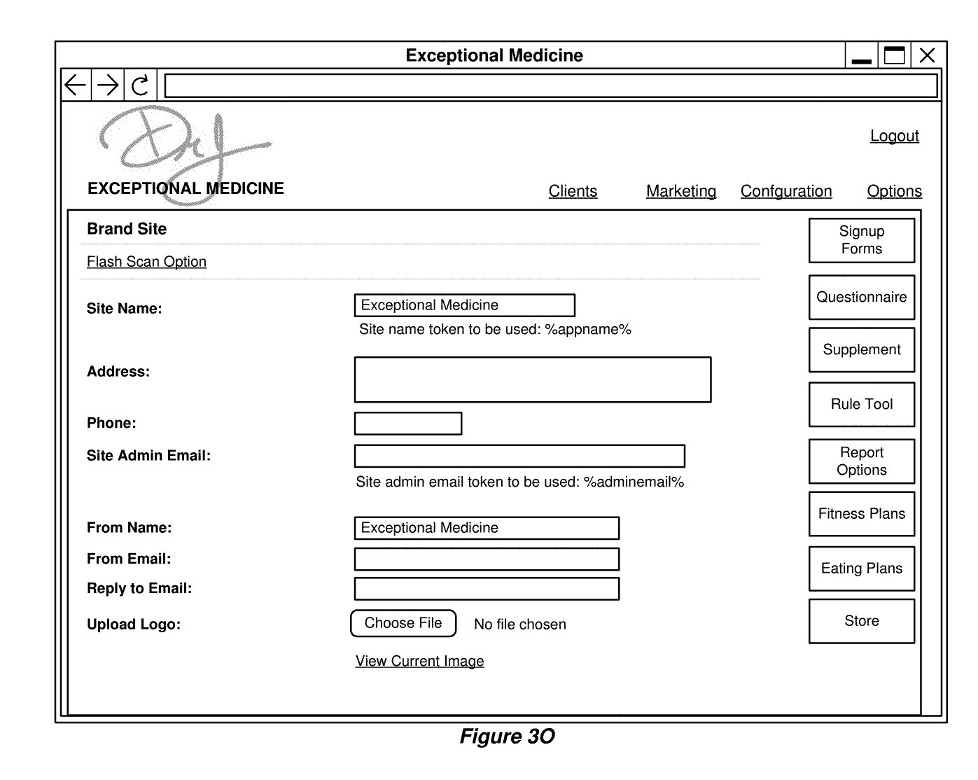

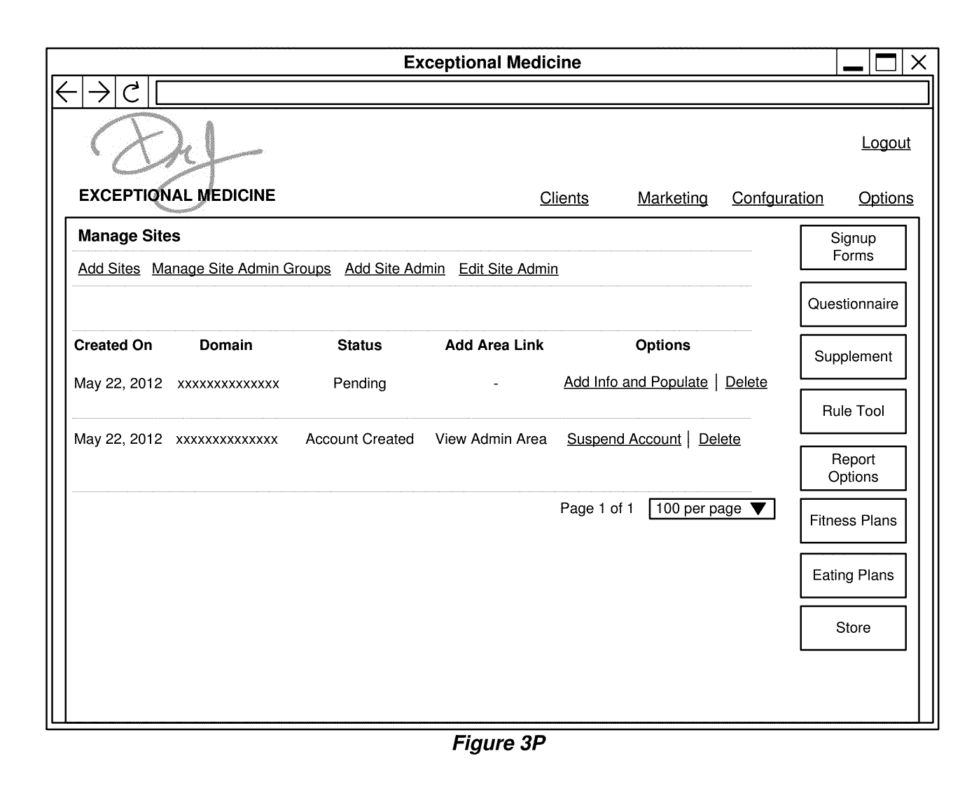

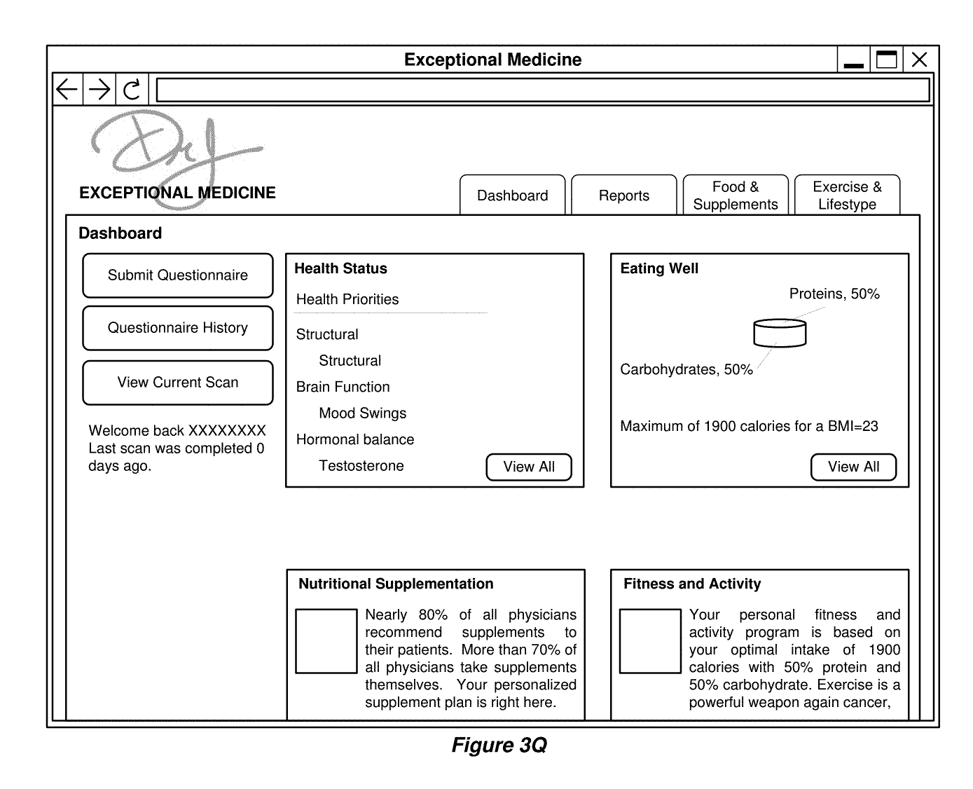

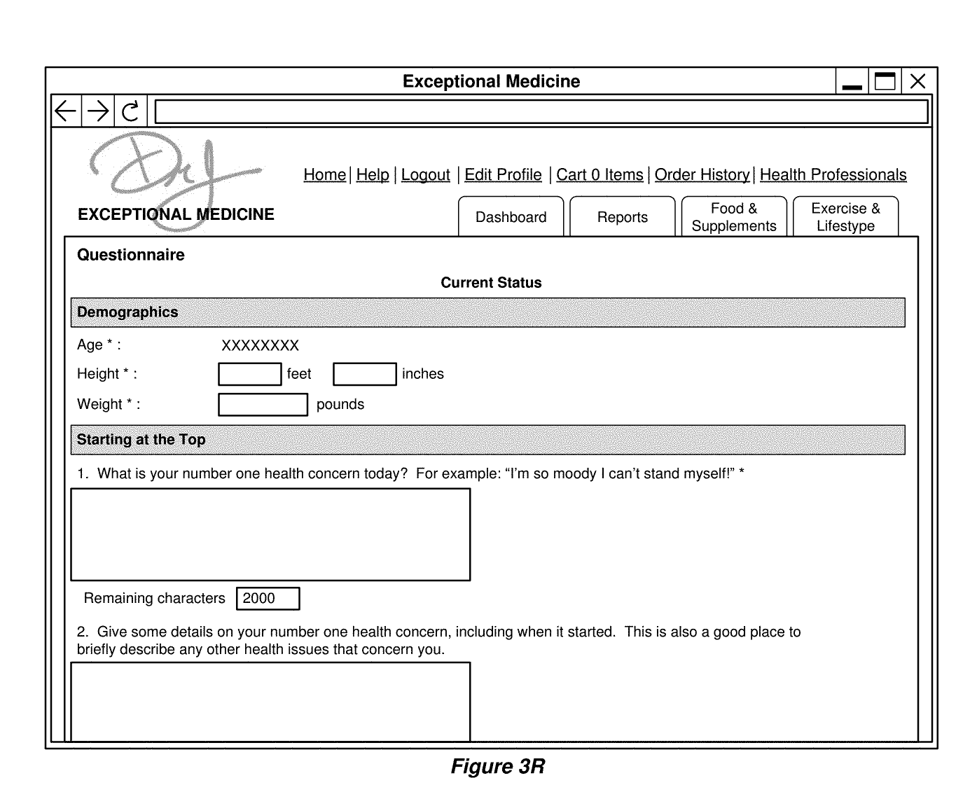

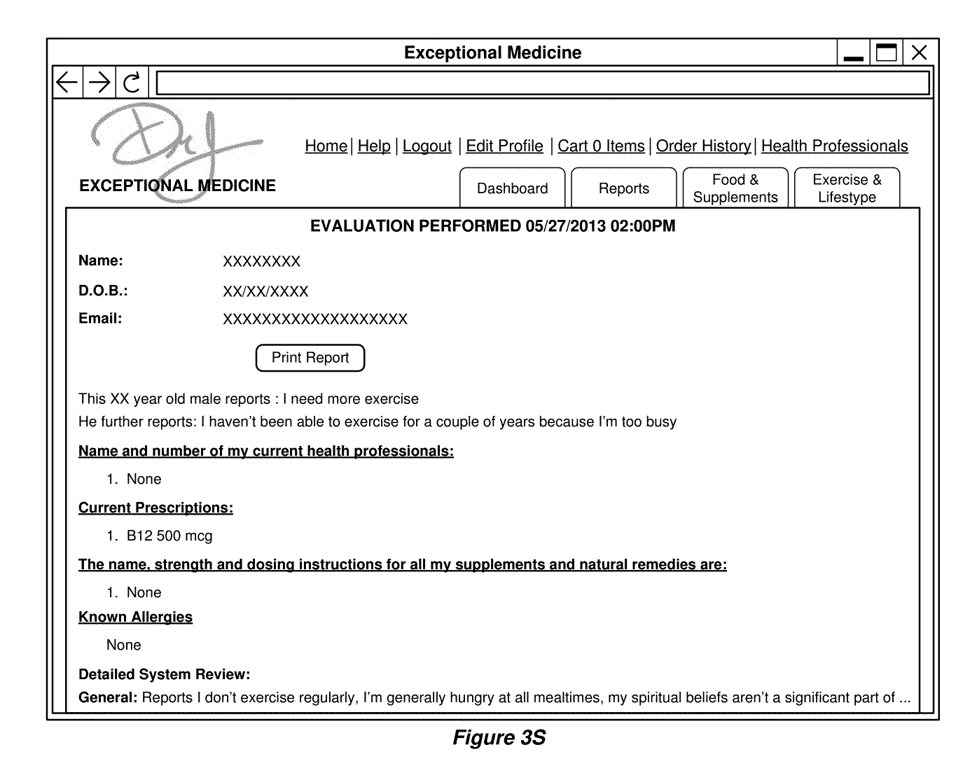

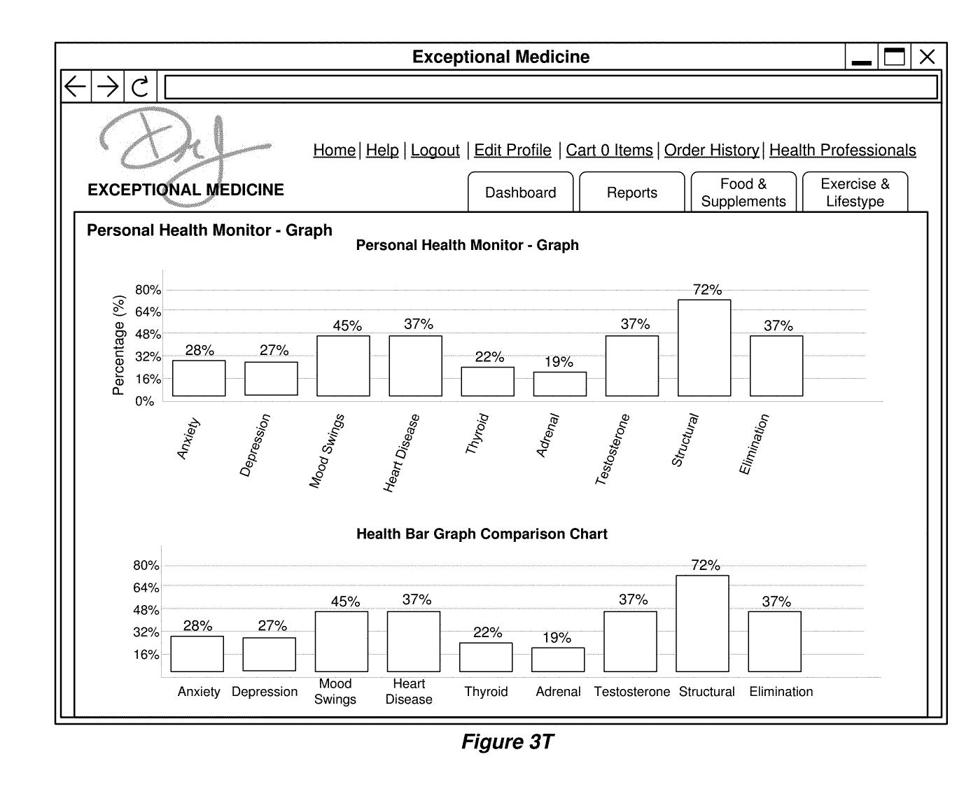

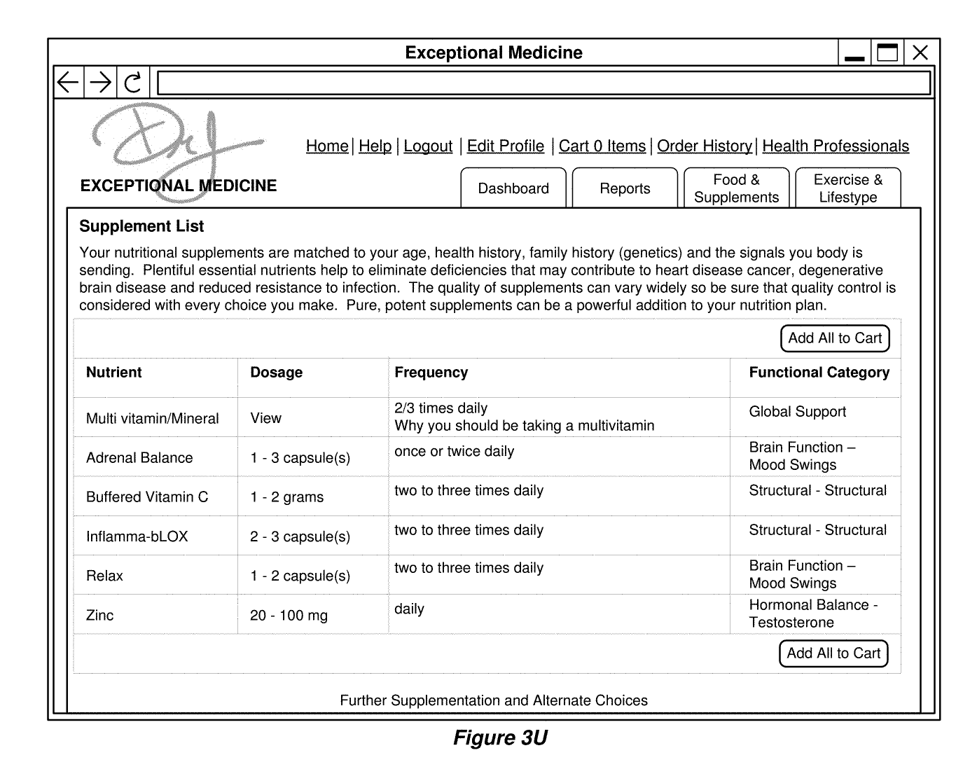

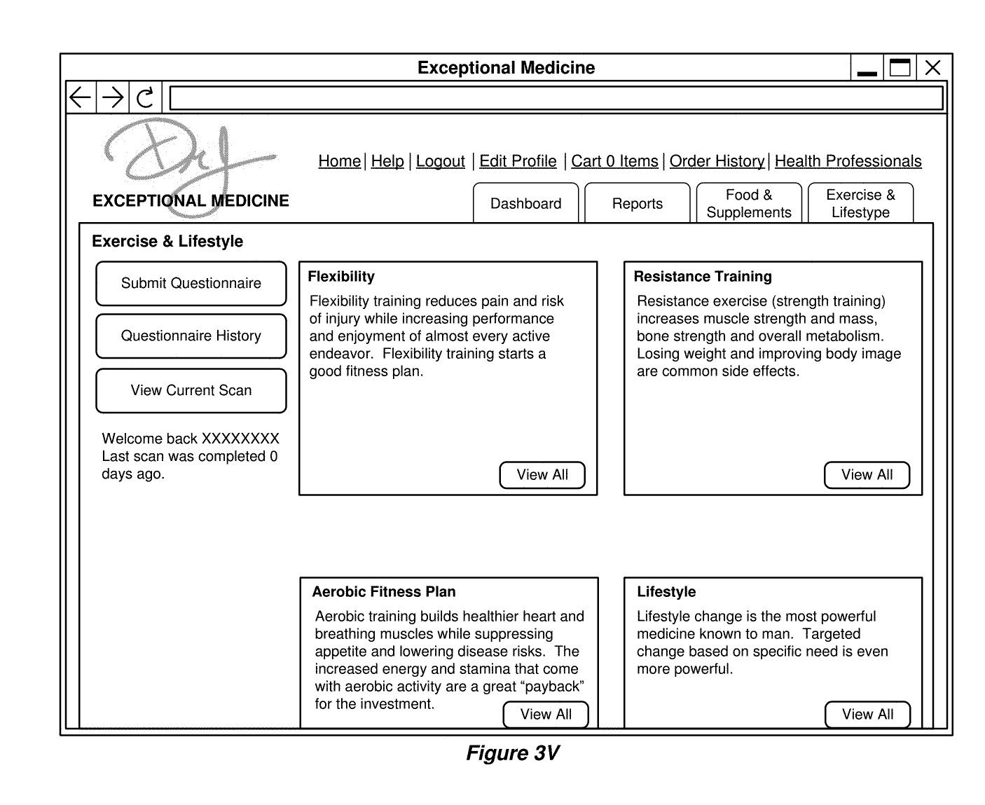

Each question is added, removed, and/or modified, at block 204. FIGS. 3A-3D show one possible non-limiting implementation providing a user interface for the purpose of adding, removing and/or modifying a question. According to this implementation, each question is user configurable. User configurable attributes of the question include without limitation the question text, the section the question relates to, the category the question relates to, the question order relative to the other questions, the type of answer in response to the question, the possible answers in response to the question (where applicable), whether the question is applicable to a male and/or a female, and whether an answer to the question is required. As shown in FIG. 3B, the type of answer in response to the question may include without a single selection (i.e., radio button) of one or more possible answers, user input (i.e, textbox), multiple selection (i.e., checkboxes), yes/no (i.e., radio button) and numbered list. If the type of answer in response to the question is a single selection or a multiple selection, then possible answers in response to the question may be defined by the user.

At block 206, each problem area (and associated minimum and maximum thresholds) is added, removed and/or and/or modified. FIG. 3W shows one possible non-limiting implementation of providing a user interface to a user adding, removing and/or modifying a problem area. According to this implementation, each problem area is user configurable. User configurable attributes of the problem area include without limitation the name of the problem area, the minimum threshold percentage, and the maximum threshold percentage, and a description of the problem area. If the functional score associated with a rule, as described above, is equal to or between the minimum and maximum threshold percentages, then the problem area will be identified as an issue for the patient or user.

Each rule is added, removed and/or modified, at block 208. FIGS. 3F-3H show one possible non-limiting implementation of providing a user interface for the purpose of adding, removing and/or modifying a rule. According to this implementation, each rule is user configurable. User configurable attributes of the rule include without limitation the rule text, the rule description, a numeric value associated with each answer for each of the questions for this rule. According to one non-limiting embodiment, the numeric value may range in value from −5 to 5. Answers which do not affect a rule are identified with a numeric value of 0. The total points for any problem area a person may have is the sum of numeric values for each of the questions. The points received for a patient during testing for a rule (by the patient answering the questions provided) is determined by adding to or subtracting from the total points for any problem area a person may have.

At block 210, the questions, problem areas and thresholds, rules, and configuration information are stored in the database 110.

The fitness and nutritional wellness determination 300 module is used to determine the fitness and wellness of the patient. At block 302, the system 100 presents a questionnaire to a patient. The responses to the questionnaire is received at block 304. At block 306, the responses to the questionnaire are mapped to the appropriate rules and a numerical functional score is calculated. The numerical functional scores outside of defined thresholds are filtered at block 308. At block 310, based on the numerical functional scores, a fitness plan, eating plan and/or supplement(s) are determined by the system 100. The numerical functional score and trend information is stored in the database 110 at block 312. At block 314, the questionnaire results are displayed. The results may be displayed as a report, a graphical chart, numerical functional scores, and the like. The user is provided a health and wellness recommendation to the patient or user 102.

The managing fitness and nutritional wellness 400 module is used to manage and maintain a patient's health and wellness. At block 402, historical information is stored in the database 110. The trends are calculated and displayed at blocks 404 and 406. At block 408, the results are displayed to the user. The results may be displayed as a report, a graphical chart, numerical functional scores, and the like.

Referring to FIG. 2E, a flow chart 500 illustrating a method for determining rules in accordance with an embodiment of the present invention, is shown. At block 502, a BMI index group is created. BMI (body mass index) group classifications include without limitation under weight, normal weight, over weight and obesity. Functional groups are created at block 504. At block 506, functional areas are created by assignment using functional groups. The functional area is then assignment to a rule tool and subsequently is what is displayed on the hot spot score chart. At block 508, problem pages are created by, without limitation, adding a rule name, assigning minimum and maximum values and adding a description. Rule tools are created using one of the previously selected problem page tool names and the functional area, at block 510. At block 512, gender, age groups and BMI areas are assigned. At block 514, the questions are displayed and assigned weighted values of positive or negative point values. The assigned weighted values of positive or negative point values are summed for each functional area based on a patient's responses to the questions. The functional area scores are then shown on a hot spot graph based on being above the minimum threshold assigned for each of the problem page areas.

Referring to FIG. 2F, a flow chart 550 illustrating a method for calculating patient scores in accordance with an embodiment of the present invention, is shown. At block 552, patient test results are store in the system. The test results are based on a test taken by the patient. The rules tool engine and correlation engine interpret the test results using without limitation (i) BMI index groups, (ii) functional groups, (iii) functional areas, (iv) problem pages, (v) rule tools from patient's answers to the questionnaire, and (vi) objective data such as blood work and x-rays, at block 554. At block 556, a hot spot score is generated based on the interpreted results from block 554 above. The system generates recommended doctor actions at block 558. At block 560, nutritional supplements are recommended.

FIGS. 3A-3W illustrate an exemplary user interface and computer program for analyzing and managing fitness and nutritional wellness in accordance with an embodiment of the present invention. It is understood that other user interfaces are possible within the scope of the invention. According to one embodiment, when an administrator logs into the system 100, the administrator is presented with a graphical user interface similar in appearance to the graphical user interface shown in FIGS. 3A-3P. This allows the administrator to configure the system 100. The menu options include, without limitation, “Clients”, “Marketing”, “Configuration”, “Options”, “Signup Form”, “Questionnaire”, “Supplement”, “Rule Tool”, “Problem Areas” (not shown), “Report Options”, “Fitness Plans”, and “Eating Plans”.

If the administrator selects “Signup Forms”, the administrator can, without limitation, manage patient signup forms and coupons.

If the administrator selects “Questionnaire”, the administrator is presented with a graphical user interface as shown in FIG. 3A. The questionnaire is separated into different health and wellness sections, including without limitation “Current Status”, “General”, “Mind and Mood”, “Circulation and Breathing”, “Digestion and Elimination”, “Body Chemistry”, “Lifestyle and Social History”, “Structural” and “At Last”. New sections may be added using this graphical user interface. The sections may be edited by selecting “Manage Questions” and using a graphical user interface similar in appearance to one shown in FIGS. 3B-3D. Using the graphical user interface, the administrator can, without limitation, modify the question text, section, category, order, and report options.

If the administrator selects “Problem Areas”, the administrator is presented with a graphical user interface like the one shown in FIG. 3W. Using the graphical user interface, the administrator can, without limitation, modify the problem area name, the minimum threshold percentage value, the maximum threshold percentage value, and the problem area description.

If the administrator selects “Rule Tool”, the administrator is presented with a graphical user interface like the one shown in FIGS. 3E-3G. Using the graphical user interface, the administrator can, without limitation, modify the rule description and the numerical functional score associated with each question.

If the administrator selects “Supplements”, the administrator is presented with a graphical user interface like the one shown in FIG. 3H. Using the graphical user interface, the administrator can, without limitation, modify supplement name, notes, warning, source, dosage, frequency, age group and gender information.

If the administrator selects “Report Options”, the administrator is presented with a graphical user interface like the one shown in FIGS. 3I-3K. Using the graphical user interface, the administrator can, without limitation, add or modify report option categories, assign report options, and assign product to questions. Report option categories include without limitation, “General”, “HEENT”, “SKIN”, “CARDIOPULMONARY”, “DIGESTIVE”, “ENDOCRINE”, “IMMUNE”, “STRUCTURAL”, “GENITOURINARY” and “NEUROPSYCH”.

If the administrator selects “Fitness Plans”, the administrator is presented with a graphical user interface like the one shown in FIG. 3L. Using the graphical user interface, the administrator can, without limitation, add or manage fitness plans, and exercises.

If the administrator selects “Eating Plans”, the administrator is presented with a graphical user interface like the one shown in FIG. 3M. Using the graphical user interface, the administrator can, without limitation, add or manage eating plans, food type, food category and food.

If the administrator selects “Store”, the administrator is presented with a graphical user interface like the one shown in FIG. 3N. Using the graphical user interface, the administrator can, without limitation, add or manage store products and shipping options.

If the administrator selects “Options”, the administrator is presented with a graphical user interface like the one shown in FIG. 3O. The menu options include, without limitation, “Brand Site”, “Store Options”, “Add Admin”, “Manage Sites”, “Update Standard Zip Files”, “Autoresponders”, and “Change Password”. If the administrator selects “Manage Sites”, the administrator is presented with a graphical user interface like the the one shown in FIG. 3P. Using this graphical user interface, the administrator add or manage sites and site administration.

According to one embodiment, when patients or users log into the system 100, the patient is presented with a graphical user interface like the graphical user interface shown in FIGS. 3Q-3V that allows the patient to access and improve their health and wellness. The menu options include, without limitation, “Dashboard”, “Reports”, “Food & Supplements”, and “Exercise & Lifestyle”.

If the patient selects “Dashboard”, the patient is presented with a graphical user interface like the one shown in FIG. 3Q. The patient can, without limitation, submit and view questionnaires, and view their current status. The patient is also provided information regarding the patient's health status, and provided recommendations regarding eating, nutritional supplements, and fitness and activity.

If the patient selects “Submit Questionnaire”, the patient is presented with a graphical user interface like the one shown in FIG. 3R. The patient can respond to the questions that have been predefined by the administrator using this user interface. Based on the responses, the patient may be provided with one or more reports or graphs using the graphical user interface like the graphical user interface shown in FIGS. 3S-3T. These reports and charts allow a patient to make improvements in their health and wellness.

If the patient selects “Food & Supplements”, the patient is presented with a graphical user interface like the one shown in FIG. 3U. This user interface provides the patient with nutritional supplement recommendations based on their responses to the questions and the predefined correlation rules defined by the administrator.

If the patient selects “Exercise & Lifestyle”, the patient is presented with a graphical user interface like the one shown in FIG. 3V. This user interface provides the patient with exercise and lifestyle recommendations based on their responses to the questions and the predefined correlation rules defined by the administrator.

It will be apparent to those skilled in the art that various modifications and variations can be made in the individualized health evaluation system and method of the present invention without departing form the spirit or scope of the invention. Thus, it is intended that the present invention cover the modifications and variations of the invention provided they come within the scope of the appended claims and their equivalents.

The present invention includes a computer program which may be hosted on a storage medium or other computer readable medium and includes instructions which perform the processes set forth herein. The storage medium or other computer readable medium can include, but is not limited to, any type of disk including floppy disks, optical disks, CD-ROMs, magneto-optical disks, ROMs, RAMs, EPROMs, EEPROMs, flash memory, magnetic or optical cards, or any type of media suitable for storing electronic instructions.

Obviously, many other modifications and variations of the present invention are possible in light of the above teachings. The specific embodiments discussed herein are merely illustrative, and are not meant to limit the scope of the present invention in any manner. It is therefore to be understood that within the scope of the disclosed concept, the invention may be practiced otherwise then as specifically described.

1. A system for serving web pages offering health, fitness and nutritional wellness information, the system comprising:
a plurality of remote computers in communication with a respective plurality of remote users;
a central server having a computer program stored in non-transient memory and one or more microprocessors;
a network interface in communication with the central server and the plurality of remote computers over a network, the network interface being configured to receive patient information;
a shared database in communication with the central server;
wherein the central server is programmed by means of the computer program to:
receive configuration and setup information, wherein the configuration and setup information includes configuration of user configurable fitness and wellness questions derived from validated questionnaires in a plurality of subspecialty fields, and user configurable correlation rules associated with the fitness and wellness questions, and wherein the user configurable correlation rules include one or more predefined thresholds;
receive and store patient information in the database, wherein the patient information includes responses to the user configurable fitness and wellness questions;
correlate the responses based on the user configurable correlation rules to a plurality of functional scores using the one or more microprocessors;
provide a health and wellness recommendation based on the functional scores; and
transmit one or more web pages, via the network interface, representing the functional scores within the user configurable thresholds, and display the one or more web pages on respective displays of one or more of the plurality of remote computers in communication with respective one or more of the plurality of remote users, wherein the user configurable threshold includes an upper threshold value and a lower threshold value, functional scores greater than the upper threshold value are assigned a standard maximum value in the health and wellness recommendation, and wherein functional scores less than the lower thresholds are not included in a final health and wellness recommendation.

2. The computer program of claim 1, wherein the central server is further configured to maintain historical patient information in the database and display the historical patient information as a graphical trend chart.

3. The computer program of claim 1, wherein the correlation rules include assigning functional scores to the user configurable fitness and wellness questions.

4. The computer program of claim 1, wherein the user is selected from at least one of the group consisting of a patient, a client of a health professional, a doctor, a health professional, an insurance administrator, a health product vendor, software developer and a system administrator.

5. A computer program product embodied on a non-transitory computer readable medium for transmitting web pages offering health, fitness and nutritional wellness information to remote users, wherein the computer program is implemented by one or more processors executing processor instructions, the computer program product comprising:
a first computer code for receiving configuration and setup information, wherein the configuration and setup information includes configuration of user configurable fitness and wellness questions, and user configurable correlation rules associated with the fitness and wellness questions, and wherein the user configurable correlation rules include one or more user configurable thresholds;
a second computer code for receiving and storing patient information in the database, wherein the patient information includes responses to the user configurable fitness and wellness questions;
a third computer code for correlating the responses based on the user configurable correlation rules to a plurality of functional scores using the one or more microprocessors;
a fourth computer code for providing a health and wellness recommendation based on the functional scores;
a fifth computer code for transmitting one or more web pages, via the network interface, representing the functional scores within the user configurable thresholds, and displaying the one or more web pages on respective displays of one or more of the plurality of remote computers in communication with respective one or more of the plurality of remote users, wherein the user configurable threshold includes an upper threshold value and a lower threshold value, functional scores greater than the upper threshold value are assigned a standard maximum value in the health and wellness recommendation, and wherein functional scores less than the lower thresholds are not included in a final health and wellness recommendation.

6. The computer program product of claim 5, wherein the user is selected from at least one of the group consisting of a patient, a client of a health professional, a doctor, a health professional, an insurance administrator, a health product vendor, software developer and a system administrator.
